(19) United States
(12) Patent Application Publication
(10) Pub. No.: US 2023/0069826 A1
(43) Pub. Date: Mar. 9, 2023

(54) NEURO ACCESS GUIDE WIRE

(71) Applicant: NEURAVI LIMITED, Galway (IE)

(72) Inventors: Karl KEATING, Galway (IE); David VALE, Barna (IE)

(73) Assignee: NEURAVI LIMITED, Galway (IE)

(21) Appl. No.: 17/940,352

(22) Filed: Sep. 8, 2022

Related U.S. Application Data
(60) Provisional application No. 63/241,881, filed on Sep. 8, 2021.

(51) Int. Cl. A61M 25/09; A61M 25/06

(52) U.S. Cl. CPC A61M 25/09041; A61M 25/06

(57) ABSTRACT

An access guidewire has a distal portion including a distal portion length and a distal portion diameter. The guidewire also includes an intermediate portion, proximal to the distal portion, having an intermediate portion length and an intermediate portion diameter. A proximal portion is proximal of the intermediate portion and includes a proximal portion length and a proximal portion diameter. The intermediate portion diameter is greater than the the distal portion diameter and the proximal portion diameter.

### CROSS-REFERENCE TO RELATED APPLICATION

The present application claims benefit of priority to U.S. Provisional Patent Application No. 63/241,881 filed Sep. 8, 2021. The entire contents of this application are hereby incorporated by reference.

### FIELD OF THE INVENTION

The present invention relates to guidewire that can be used to hold or expand a large bore catheter while accessing the neurovascular.

### BACKGROUND

Guidewires are known in the art along with large bore catheters. However, passing a large bore catheter over a guidewire to its location within the neurovascular can be challenging given the size differential. Typically, an intermediate catheter is used that is sized between the devices to help with transition or opening the distal end of the large bore catheter. Using a second catheter can be a waste of resources and also time as the user needs to advance the intermediate catheter prior to the large bore catheter.

### SUMMARY

Examples of the present invention of an access guide wire that can have a section that includes larger outer diameter (OD) portion to bridge gap between guidewire OD of 0.014 inch or 0.022 inch and a large bore catheter inner diameter (ID) of 0.070 inch. These sizes are important when accessing the distal neurovascular, there is a gap between the ID of large bore catheters and guidewire ODs used in gaining access to distal cerebral vessels such as the ICA and the M1/M2.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 illustrates a side view of the access wire of the present invention.

FIG. 2 illustrates a side view of the access wire with a deflected distal end.

FIG. 3 illustrates a cross-section view of another example of the access wire.

FIG. 4 illustrates a cross-section view of further example of the access wire.

FIG. 5 illustrates a cross-section view of yet another example of the access wire.

FIG. 6 illustrates a side view of another example of the access wire.

FIG. 7 illustrates a partial cross-section view of the access wire inside a large bore catheter.

FIG. 8 illustrates a cross-section along line X-X of FIG. 7.

### DETAILED DESCRIPTION

Specific examples of the present invention are now described in detail with reference to the Figures, where identical reference numbers indicate elements which are functionally similar or identical. Accessing the various vessels within the vascular, whether they are coronary, pulmonary, or cerebral, involves well-known procedural steps and the use of a number of conventional, commercially-available accessory products. These products, such as angiographic materials, rotating hemostasis valves, and large and intermediate catheters are widely used in laboratory and medical procedures. When these products are employed in conjunction with the system and methods of this invention in the description below, their function and exact constitution are not described in detail. While the description is in many cases in the context of neurovascular treatments, the systems and methods may be used for other procedures and in other body passageways as well.

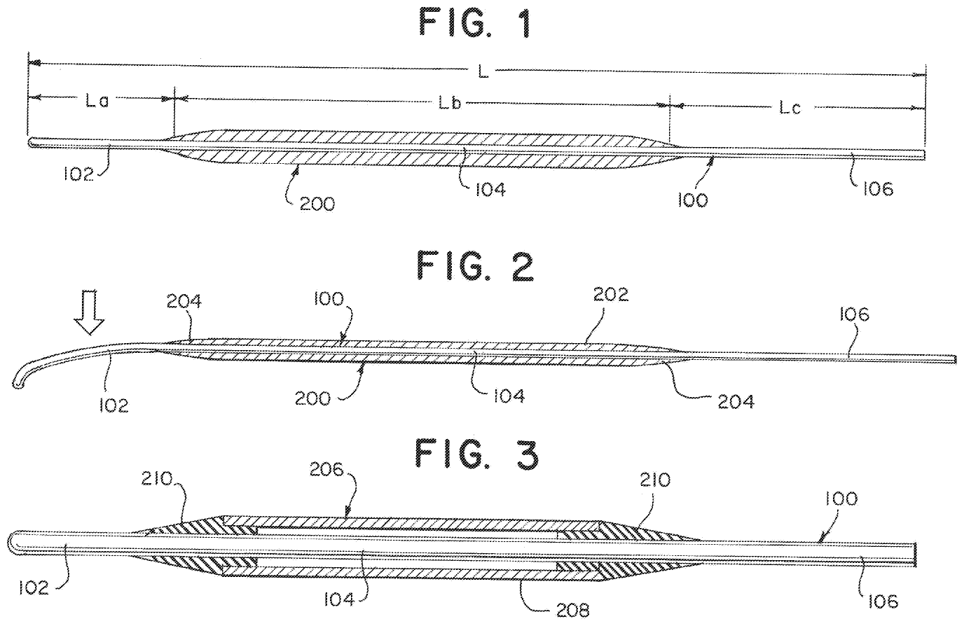

FIG. 1 illustrates an example of an access guidewire 100 of the present invention. A typical prior art guidewire has a uniform diameter across its length. The access guidewire 100 has a length L, a distal portion 102, and intermediate portion 104, and a proximal portion 106. The distal portion 102 can have a length La, the intermediate portion 104 has a length Lb and the proximal portion has a length Lc. The intermediate portion 104 can have an increased diameter ΦB in relation to the distal portion diameter ΦA and the proximal portion diameter ΦC (see FIG. 6). In FIGS. 1 and 2 the distal portion diameter ΦA and the proximal portion diameter ΦC can be equal while the intermediate portion diameter ΦB is larger. The increased diameter portion 200 can be same material as the rest of the guidewire 100, just formed with a larger circumference/diameter.

FIG. 2 illustrates that the distal portion 102 can retain its flexibility along its length La as is typical for a guidewire. The increased diameter portion 200 can have reduced flexibility or retain flexibility consistent with the distal portion 102. In this example, the proximal portion 106 can be the portion proximal the increased diameter portion 200 and extend to the operator. The flexibility of the proximal portion 106 can mirror that of a typical guidewire or have differential flexibility along its length as may be necessary to deliver it to the neurovascular region of a patient.

FIG. 1 illustrates an example where the intermediate portion 104 and the increased diameter portion 200 are made of the same material. FIGS. 2, 3, 4, and 5 illustrate examples where the increased diameter portion 200 can be separately provided on the intermediate portion 104.

FIGS. 2, 3, 4, and 5, illustrate an example where the access guidewire 100 has a uniform diameter across the distal portion 102, the intermediate portion 104, and the proximal portion 106. Instead, as in FIG. 2, the increased diameter portion 202 can be formed over the intermediate portion 104. In one example, the increased diameter portion 202 can be a soft reflowed jacket over the guidewire 100 with tapered ends 204.

FIG. 3 illustrates that the increased diameter portion 206 can have an outer tube 208 or composite tube which can also have a coil/braid support. The tapered ends 210 can be the adhesive bond that holds the outer tube 208 in place and is filleted to allow for a smooth transition. In one example, the outer tube 208 can be made of Pebax 25D or 35D or Neusoft Jacket 42A/62A.

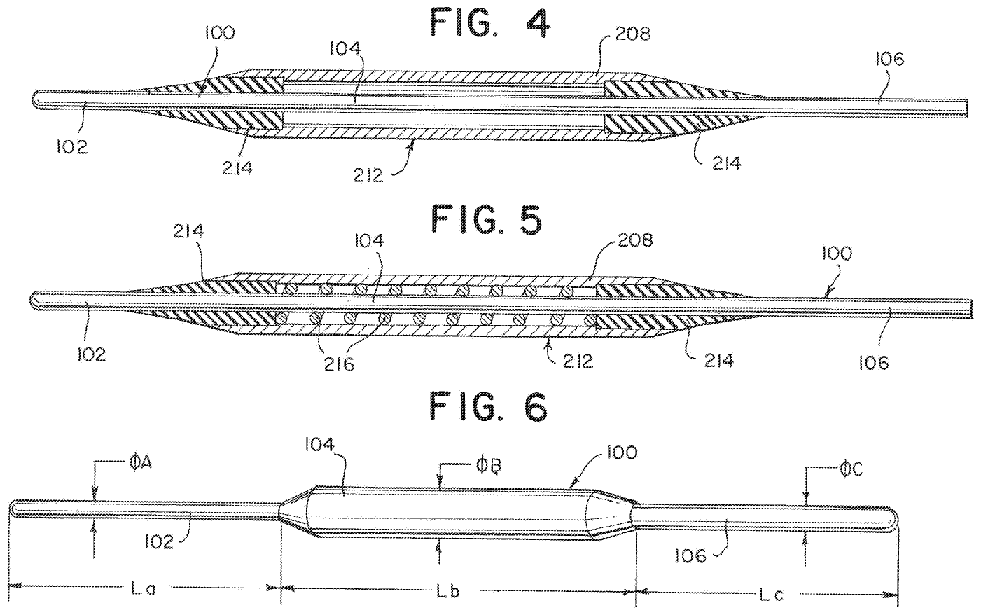

FIG. 4 illustrates a two-tube example for the increased diameter portion 212. Here the outer tube 208 is placed over an inner tube 214 and the inner and outer tubes 214, 208 are reflowed to form the increased diameter portion 212. In this example, the outer tube 208 can be made of Pebax 25D or 35D while the inner tube 214 can be made of Neusoft 42A/62A.

FIG. 5 illustrates a variant example of FIGS. 3 and 4, here a coil 216 is placed inside the outer tube 208 to provide strength and kink resistance. Note that in the variant of increased diameter portion 212 of FIG. 4, the outer tube 208 and the inner tube 214 are reflowed to have tapered profile. The inner coil 216 offers kink resistance with thin flexible outer tubes 208. The inner coil 216 can be free floating or the outer tube 208 can be reflowed through it. The inner coil 216 can be replaced with a braid (not illustrated).

FIG. 6 illustrates an example where the distal section 102 can have smaller diameter ΦA (i.e. a lower profile) than the proximal section 106 (ΦC). Thus, the proximal portion 106 can be thicker for greater pushability and handling, with a smaller distal section/tip 102 for atraumatic clot crossing.

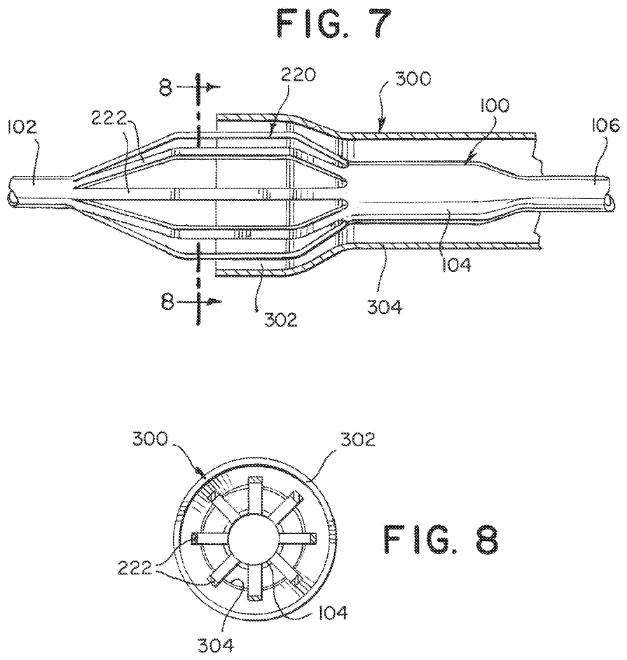

FIGS. 7 and 8 illustrate an example of the increased diameter portion 220 with a stepped, fluted mid-section 222. Fluted mid-section 222 can be used to provide a smooth transition when used with a funnel catheter 300 to help expand the tip 302 of the catheter 300. The flutes 222 can be thin and soft so that they can be collapsed and retrieved through the body 304 of the funnel catheter 300.

In specific, non-limiting, examples:
the length L of the guidewire 100 can be 180 cm;
the length La of the distal portion 102 can be 3-5 cm, as low as 1 mm or as high as 200 mm, two examples: a short 2 cm shapeable tip, and another with a long 15 cm tip, of which the most distal approximate 2 cm is shapeable;
the distal portion (outer) diameter ΦA can be 0.014-0.022 inch and as low as approximately 0.010 inch or as high as 0.038 inch, one range can be about 0.010 inch to about 0.018 inch range, and examples can be 0.014 inch;
the proximal portion length Lc can depend on lengths La and Lb, as overall device length L needs to be longer than the catheter 300 it is used with, in one example, to be a few cm longer than the overall length of the funnel catheter 300, which is approximately 130 cm; and
the proximal portion (outer) diameter ΦC can be 0.014-0.022 inch, as high as 0.045 inch and as low as 0.014 inch, and examples are about 0.035 inch.

Turning to examples of the intermediate/increased diameter portion 104, 200, 202, 208, 220:
the length Lb can be 200-300 mm, as short as 2 cm, or as long as the entire device (minus the tip length La), in one example, about 20 cm;
the (outer) diameter ΦB can be 0.065 inch; and
the (outer) diameter ΦB can depend on the inner diameter (ID) of the catheter 300 that is being used in conjunction with, and can be approximately 0.003 inch to 0.006 inch less than that ID.

Examples of the device 100 can be used with a large bore intermediate catheter (0.068 inch-0.072 inch ID approximately), a superbore catheter (0.080 inch-0.092 inch approximately), or a funnel catheter (shaft ID of 0.068 inch-0.072 inch approximately).

The invention is not necessarily limited to the examples described, which can be varied in construction and detail. The terms “distal” and “proximal” are used throughout the preceding description and are meant to refer to a positions and directions relative to a treating physician. As such, “distal” or “distally” refer to a position distant to or a direction away from the physician. Similarly, “proximal” or “proximally” refer to a position near to or a direction towards the physician. Furthermore, the singular forms “a,” “an,” and “the” include plural referents unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

In describing example embodiments, terminology has been resorted to for the sake of clarity. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Some steps of a method can be performed in a different order than those described herein without departing from the scope of the disclosed technology. Similarly, it is also to be understood that the mention of one or more components in a device or system does not preclude the presence of additional components or intervening components between those components expressly identified. For clarity and conciseness, not all possible combinations have been listed, and such modifications are often apparent to those of skill in the art and are intended to be within the scope of the claims which follow.

1. An access guidewire, comprising:
a distal portion comprising a distal portion length and a distal portion diameter;
an intermediate portion, proximal to the distal portion, comprising an intermediate portion length and an intermediate portion diameter; and
a proximal portion, proximal of the intermediate portion, comprising a proximal portion length and a proximal portion diameter;
wherein the intermediate portion diameter is greater than then the distal portion diameter and the proximal portion diameter.
